(19) United States
(12) Patent Application Publication
(10) Pub. No.: US 2026/0275035 A1
(43) Pub. Date: Sep. 17, 2026

(54) One-Pot Process for Preparing Cold-Processable and Viscosity Building Composition for Personal Cleansing Products

(71) Applicant: Galaxy Surfactants Ltd., Navi Mumbai (IN)

(72) Inventors: Nirmal Koshti, Piscataway, NJ (US); Bhagyesh Jagannath Sawant, Palava City, Dombivli (E) (IN); Bilal Momin, Bhiwandi (IN); Parag Narendra Savla, Bhiwandi (IN); Juilee Jitendra Parab, Asangaon (IN)

(21) Appl. No.: 19/562,916

(22) Filed: Mar. 11, 2026

(30) Foreign Application Priority Data
Mar. 11, 2025 (IN) 202521021985

(51) Int. Cl. C08G 65/48; A61K 8/86; A61Q 5/02; A61Q 19/10; C08K 5/41

(52) U.S. Cl. CPC C08G 65/48; A61K 8/86; A61Q 5/02; A61Q 19/10; C08K 5/41

(57) ABSTRACT

Disclosed herein is a one-pot process for preparing a cold-processable and rheology modifying more particularly viscosity-building composition for personal cleansing products. The one-pot process enables to manufacture the viscosity-building composition with higher active content and ease of incorporation into personal cleansing products. The present invention also relates to such viscosity-building composition. The viscosity-building composition of the present invention comprises, fatty acid esters of polyethylene glycol (Formula I) and an anionic surfactant, N-acyl methyl taurate (Formula II). Achieving the desired viscosity at ambient temperature in case of personal care formulations like shampoos, body-washes, creams, etc. saves both energy and time spent on heating and cooling of humungous sized batches (typical commercial batch size of several metric tonnes), resulting in higher productivity and lower cost.

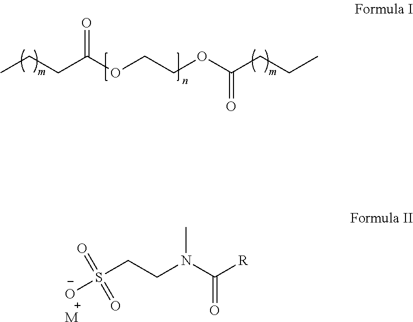

### FIELD OF THE INVENTION

The present invention relates to a process of making a cold-processable viscosity-building composition for personal care products and such viscosity building composition itself. More particularly, the process of making the composition is a one-pot synthesis and is suitable for personal cleansing products based on ‘sulphate-free’ surfactants.

### BACKGROUND OF THE INVENTION

There are several types of water containing personal care formulations/products like creams, lotions, body washes, intimate hygiene washes, shampoos and face washes. Each formulation type has its own physical attributes. These compositions need to deliver the intended benefit to the customer while being aesthetically appealing. Thickeners or viscosity-builders play an important role in both aspects of formulation, the physical attributes and the functional role. For example, a viscous cleansing formulation is not only aesthetically appealing (viscous material is always perceived as luxurious and rich i.e. concentrated) but it also helps in long term stability (emulsification, and separation or sedimentation of ingredients during storage is prevented by viscous form of the composition) of the ingredients in a cleansing formulation and it also helps in smooth delivery from the bottle (container) with or without dispenser. In case of ‘leave-on’ application like cream, in addition to the stability of formulation by way of emulsification, smooth and even delivery of ‘the active’ (anti-acne agent, UV absorbers, moisturizers) on the body surface is important. Also, in case of a cream or a lotion type of formulation, the consumer-desired sensorial aspects are enhanced by the viscosity boosters-cum-emulsifiers.

There are several types of thickeners or rheology modifiers. All viscosity boosters are macromolecules (polymeric) and these can be based on naturally occurring gums or modified gums (guar, cellulose, acacia, starch and many other carbohydrates that are available in nature) or these macromolecules can be totally synthetic, based on small molecular weight monomers such as acrylic esters and acrylic amides that are polymerized.

Naturally occurring gums are large molecular weight carbohydrates and are neither oil soluble nor water-soluble though they can swell in water. However, the swollen particles do not form one homogeneous phase in the formulation and are unstable and aesthetically unpleasant. To make them amenable to personal care formulations, usually some degree of derivatization/functionalization is done using synthetic chemistry (example, guar hydroxypropyl trimonium chloride, and hydroxy ethyl cellulose). Natural gums are biodegradable but after undergoing the functionalization by synthetic chemistry results in compromising the extent of the biodegradability.

The personal care industry widely uses acrylate polymers and copolymers as suitable viscosity-building agents. However, there are few concerns regarding its origin, safety, and environmental effects. In dermal irritation and sensitization study on humans, some acrylates copolymers were found to be weak irritants and produce slight erythema. Also, the residual monomer has the potential to induce sensitization. They are also found to cause moderate to severe eye irritation in ocular irritation studies on rabbits (Cosmetic Ingredient Review, Amended Safety Assessment of Acrylates Copolymers as Used in Cosmetics, January 2019).

Synthetic polymers, based on acrylic chemistry, had been popular but currently these are certainly not being looked at with the same degree of favor because of biodegradability issue that arise due to high degree of polymerization and cross linking of the polymeric chains. (Duis et al. Environ Sci Eur (2021) 33:21).

Though acrylate-based polymers and co-polymers are well known in cosmetic and personal care industry for thickening property, the personal care industry still needs other sustainable and safe options for viscosity-building.

On this backdrop, some of the synthetic polymers made from green monomers are useful such as polyglyceryl esters of fatty acids or esters of polylactic acid with fatty acids. Fatty acid esters of polyethylene glycols are quite popular with the personal care industry. These macromolecules are amenable to modulate hydrophobicity and hydrophilicity. One can control the repeating units of oxyethylene unit for hydrophilicity and the varying alkyl chains of fatty acids offers the scope to change hydrophobicity. One such example is, as Polyethylene glycol 150 distearate (CAS No 9005-08-7), a commonly used viscosity-builder. Polyethylene glycols (PEGs) are widely used for making non-ionic surfactants since HLB is tunable. For nonionic surfactants with PEG, the applications range from detergents, agrochemicals, food industry and many more. Polyethylene glycols up to molecular weight of 20,000 are completely biodegradable (Fusako Kawai, https://doi.org/10.1002/3527600035.bpol9012). Polyethylene glycols of lower to higher molecular weights are also used as intermediates in the preparation of resins such as alkyd resin and polyurethane resin, and as components in the manufacture of lubricants, antifreeze agents, wetting agents, printing inks, adhesives, shoe polish, softening agents, sizing agents, and plasticizers. In addition, PEGs have been used as resin gels that are used to immobilize enzymes or microbial cells, and for the chemical modification of enzymes. In summary, PEGs are biodegradable and their esters with fatty acids are biodegradable. This is one reason why PEG 150 distearate is used widely for boosting viscosity and for its emulsifying action. However, the major drawback for this kind of solid material is that it is difficult to formulate, since it needs to be mixed well with rest of the formulation and that is possible only if it is melted at elevated temperature of the mass (U.S. Pat. No. 9,937,119). Increasing temperature of whole batch is very energy intensive. Also, the time taken for heating and gradual cooling under stirring results in prolonged batch time and hence a significant drop in productivity.

In view of large scale of operations in manufacturing of personal cleansing products, there is a definite need for energy-efficient ingredients and energy-efficient processes for not only significant saving of energy but also for increased productivity and accordingly economize the process.

### OBJECTIVE OF THE INVENTION

An objective of the present invention is to provide an energy efficient method of manufacturing viscosity-building composition for personal cleansing formulation.
Another objective of the present invention is to provide a viscosity-building composition that performs efficiently at ambient temperature while manufacturing end personal cleansing formulations wherein it saves on energy and time by avoiding the heating and cooling of the entire mass of personal cleansers, thus resulting in higher productivity at lower cost.
Another objective of the present invention is to provide viscosity-building composition that allows the incorporation of heat-sensitive personal care actives into the cleansing formulations.
Yet another objective of the present invention is to provide viscosity-building compositions for personal cleansing formulations that are based on ‘sulphate-free’ gentle surfactants, particularly amino acid surfactants.
Yet another objective of the present invention is to provide a biodegradable and cold-dispersible viscosity-building composition for personal cleansers.

### SUMMARY OF THE INVENTION

To address at least one of the problems described in the background and achieve at least one of the objectives, the present invention provides, in one aspect, a one-pot process of preparing a cold-processable viscosity-building composition comprising steps of:
a. heating polyethylene glycol with a fatty acid at a temperature of 140-150° C. to prepare a fatty acid diester of polyethylene glycol of Formula I,

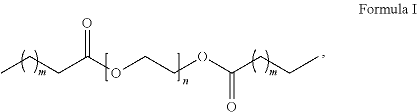

wherein m=9 to 15 and n=100 to 150;
b. cooling the fatty acid diester of polyethylene glycol obtained in step (a) to a temperature of 90-100° C., adding an N-acyl methyl taurate, of Formula II, in powder form and stirring the reaction mixture to obtain a homogeneous mass,

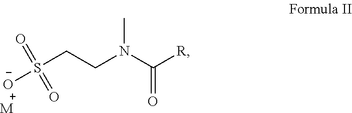

wherein R═C₇ to C₁₇ and M⁺=Na⁺ or K⁺;
c. cooling the homogeneous mass to a temperature of 70° C. to 80° C. and pouring onto a double drum flaker to obtain the cold-processable viscosity-building composition in flake form,
wherein thickness of the flake is maximum 1.0 mm and length of the flake is maximum 10 mm, and
wherein 1% homogeneous dispersion of the flakes in water is formed in 15 to 30 minutes of stirring at 25° C.

In an another aspect, the present invention provides a cold-processable viscosity-building composition comprising:
a. a fatty acid diester of polyethylene glycol of Formula I

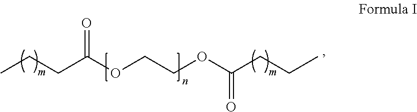

wherein m=9 to 15 and n=100 to 150; and
b. an N-acyl methyl taurate of Formula II

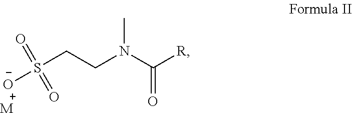

wherein R═C₇ to C₁₇ and M⁺=Na⁺ or K⁺,
wherein the weight ratio of the fatty acid diester of polyethylene glycol to the N-acyl methyl taurate is between 0.7:0.3 and 0.9:0.1,
wherein the viscosity-building composition is in a flake form,
wherein the thickness of the flake is maximum 1.0 mm and length of the flake is maximum 10 mm, and
wherein 1% homogeneous dispersion of the cold-processable viscosity-building composition in water is formed in 15 to 30 minutes of stirring at 25° C.

### BRIEF DESCRIPTION OF THE FIGURES

FIG. 1: Depicts the measurement of the length of the flakes of viscosity-building composition of the present invention.

### DETAILED DESCRIPTION OF THE INVENTION

Personal care products are meant to be applied to the external parts of the body. Personal cleansers are used every day by one and all, by the entire human population and several million tons are produced every day. Examples of such big volume personal cleansers are shampoos, body-washes, hand-washes, facewashes, intimate hygiene washes.

Personal cleansers have evolved over the last two decades. The traditional soap and other harsh surfactants are getting phased out due to high alkaline pH and excessive removal of lipids/proteins of stratum corneum or hair surface, resulting in dryness of skin and hair which leads to further complications. This advancement in personal cleansers is the result of understanding of interaction between surfactants and the constituents of skin/hair. This advancement in personal cleansing science led to the rise of gentler yet effective surfactants particularly, the amino acid surfactants, wherein amino group of an amino acid is acylated to create the hydrophobic portion of the surfactant. Mild surfactants like amino acid surfactants and a few other ‘sulphate-free’ surfactants are the most preferred anionic surfactants for personal cleansers. However, using it in traditional forms of a classical shampoo or a body wash is challenging. Developing reasonable viscosity in cleansing formulations using amino acid-surfactants is a challenge, and thereby both important attributes of stability and aesthetics of the formulations are lost as explained in the background section. This challenge is overcome to some extent by polymeric rheology modifiers. Of all types of available rheology modifiers, synthetic, semisynthetic or natural, the most economical and biodegradable are the polyethylene glycol (PEG) diesters (Formula I). Unlike many other polymeric rheology modifiers, PEG diesters are effective in building the viscosity and transparency to formulations designed with mild/skin friendly amino acid surfactants at skin pH (5.0 to 6.0).

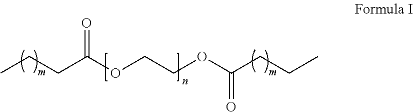

PEG diesters (Formula I) are waxy solids with significantly high melting points. (For example, PEG 150 distearate has melting point of 58-60° C. Similarly, PEG 150 hydrogenated jojobate also melts around same temperature (jojoba seed fatty acid has around 70% 11-eicosenoic acid with 20 carbon and a double bond at C11 position which is hydrogenated before converted to 150 PEG esters). PEG 150 dilaurate starts melting at 52° C.

The major drawback with PEG diesters as rheology modifiers used in the prior art is that these solid waxy products do not get dispersed at room temperature in water or in a typical mild surfactant mix for making a personal care product like body wash or shampoo.

Personal cleansers are high volume products because of huge consumption by human population for cleansing of skin, the largest organ in human body.

Though the viscosity-builders form a very small part of the cleanser formulations (1 to 3%), the whole mass of the formulation needs to be heated to 70-75° C. for their uniform dispersion in the surfactant matrix, as descried in the background and prior art, and then it is cooled down to add other thermosensitive ingredients to finally form the personal care product.

This problem could be partially addressed by converting the solid waxy viscosity builders into cold-processable liquid form by emulsification using additional water and surfactants. (U.S. Pat. No. 6,165,955 by Rhodia Inc., commercial products by Clariant Perlogen SF 117 and Perlogen SF 3000), however, the pearly wax content in the emulsion is around 20 to 25% in those room temperature processable pearlizer. Often such emulsions are not stable, and shelf-life is very limited due to runaway increase in viscosity over a few weeks of storage at temperatures of 40° C. (Handling and storage instructions in globally harmonized SDS of such emulsified pearly wax, Sparkle 655 by Galaxy Surfactants Ltd., India).

The present invention addresses the limitation of cold processibility by providing an energy efficient process and energy efficient incorporation of the cold processable viscosity building composition into final personal cleanser like a shampoo or a body wash. More particularly, the present invention provides a process of preparation of viscosity building composition wherein at high temperature incorporation of N acyl methyl taurate is carried out during formation of fatty acid diester of PEG, and flaking final composition. By doing so, the present inventors have found that the heating step, which is later required to incorporate viscosity builders, while preparing personal care formulations, is eliminated.

The present invention provides a method of making viscosity-building composition comprising of PEG diesters of fatty acid (Formula I, wherein m=9 to 15 and n=100 to 150) and sodium N-acyl methyl taurate (Formula II, wherein R═C₇ to C₁₇ and M⁺=Na⁺ or K⁺).

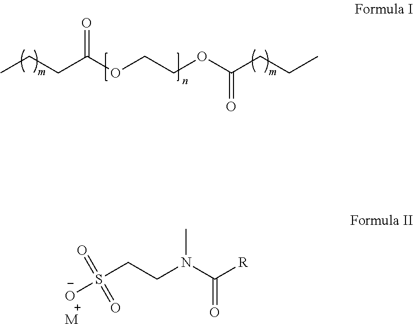

Polyethylene glycol diesters of Formula I:

Polyethylene glycols diesters are made by esterification of glycols with fatty acids. Polyethylene glycols of the present invention are selected from polyethylene glycols with molecular weight range of 4000 to 6000 (CAS No. 25322-68-3). Fatty acids that are used for esterification with polyethylene glycol are derived from vegetable oil with carbon chains ranging from C₈ to C₁₈. However, PEG 150 hydrogenated jojoba fatty acid (CAS No. 329360-70-5) based, is commercially available which has 75% of C₂₀ alkyl chain. There are several oils like Mustard seed oil that has C₂₂ carbon erucic acid and the saturated fatty acids particularly form vegetable origin are suitable for viscosity builder derivatives of PEG. Esterification of pure single fatty acid or mixtures of fatty acids that are commercially available can be done. Fatty acid derived from vegetable sources can be used as such with their naturally occurring unsaturation or they can be used after saturating by hydrogenation.

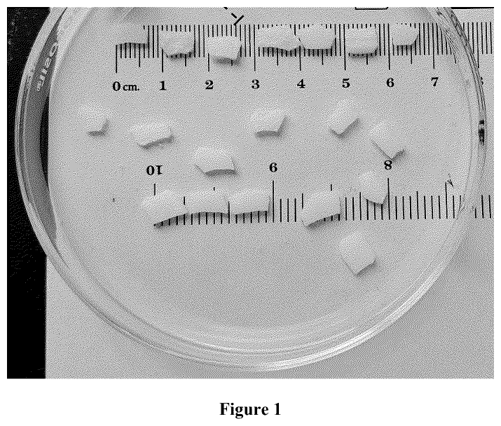

The PEG diester of fatty acids are prepared by esterification reaction of fatty acid and polyethylene glycols. Acid catalyzed esterification is carried out at 140 to 160° C. in presence of hypophosphorous acid as catalyst. The progress of esterification is monitored by drop in acid value. The molten mass of the PEG diester is cooled to 100° C. and to it, sodium N-acyl methyl taurate (Formula II) in powder form (particle size <200μ) is added under stirring and continued to cool to 80° C. The resultant homogeneous mass is flaked on the double drum flaker to get thickness of flakes of 1 mm maximum and the length of the flakes, 10 mm maximum (FIG. 1). For thickness measurement, a micrometer is adjusted to a 1 mm gap between Anvil and Spindle and flakes according to present invention passes through the 1 mm gap of micrometer.

Preferably, the cold processable viscosity-building composition of the present invention comprises fatty acid diesters of PEG in an amount of around 70% to 90% by weight of the viscosity-building composition of the present invention. The fatty acid diesters of PEG forms at least 0.7 parts to 0.9 parts of total cold-processable viscosity-building composition.

In preferable embodiment, the fatty acid diesters of PEG are selected from PEG-150 distearate, PEG-150 dilaurate, PEG-150 dimyristate, PEG-150 dipalmitate. PEG-100 dilaurate, PEG-100 distearate, PEG-100 dimyristate, PEG-100 dipalmitate or mixture thereof.

In a few embodiments, the cold-processable viscosity-building composition may also comprise other derivatives of PEG diesters where molecular weight of PEG may vary from 4000 to 8000. In yet another embodiment, other viscosity boosting molecules can be the part of the viscosity-building composition of present invention. Such other viscosity boosting molecules may comprise of PEG-150 pentaerythrityl hydrogenated glyceryl palmitate, polyglyceryl monoester of fatty acid or polyglyceryl polyester of fatty acids.

### N-Acyl Methyl Taurates of Formula II:

N-Acyl methyl taurates are widely used mild anionic surfactants that are available as aqueous solution as well as solids with active matter of 90-97%. (Examples are, sodium N-lauroyl methyl taurate (CAS No 4337-75-1), sodium N-cocoyl methyl taurate (CAS No 61791-42-2), and Sodium N-oleoyl methyl taurate (CAS No 7308-16-9).

Sodium N-cocoyl methyl taurate has been described recently as a biosurfactant (S. Narasimhan et al., Cosmetics and Toiletries, July 1st issue, 2020) probably because both raw materials, coconut fatty acids and N-methyl taurine, are part of living species but commercially is made by reacting methyl amine with sodium isethionate. N-acyl methyl taurates are commercially made by chemical synthesis (fatty acid and N-methyl taurine). In addition to mildness towards the skin and hair, N-acyl methyl taurates are stable in both acidic and alkaline environment and they foam and lather well. Thus, N-acyl methyl taurates have all the desired attributes for personal cleansing.

In an embodiment, the cold processable viscosity-building composition of the present invention comprises N-acyl methyl taurate in an amount of around 10% to 30% by weight of the total composition. The surfactants form at least 0.1 parts to 0.3 parts of the total cold-processable viscosity-building composition.

In the most preferable embodiment, the N-acyl methyl taurate is sodium N-lauroyl methyl taurate, sodium N-cocoyl methyl taurate or mixture thereof.

### One-Pot Process of Preparing Viscosity Building Composition.

As described above, PEG diester of fatty acid is made at a temperature of 140 to 160° C. under the influence of acidic catalysis and mild vacuum to remove the water of esterification. The heated content of the molten ester is further utilized to incorporate the anionic surfactant, sodium N-acyl methyl taurate in powder form (particle size <200μ, moisture content less than 1.0%), and are mixed at 95 to 100° C. for 2 hours. After achieving homogeneous mass, the mass is further cooled to 80° C. The molten mass of the ester of Formula I and surfactant of Formula II is then processed using a double drum flaker. The double drum flaker comprises of two drums that are rotated in the opposite direction to each other. The frequency of rotation of the double drum is set to 1-3 rpm (depending on the drum size), and the gap between the two drums is adjusted to 0.5 mm. The Cooling/Chilling water is applied to the drums, followed by feeding the homogenous mass and allowing the material to flake. The temperature of the homogeneous molten mass is maintained at 80° C. during the flaking process. The technique (double drum flaker) is amenable to high throughput and affords the cold-processable viscosity building composition in very high active form, 80-90% of PEG diester.

In an embodiment, the present invention provides a one-pot process to prepare a viscosity-building composition, wherein the process comprises the following steps:
a. heating polyethylene glycol with a fatty acid at a temperature of 140-150° C. to prepare a fatty acid diester of polyethylene glycol of Formula I (where m=9 to 15 and n=100 to 150);

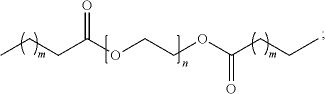

b. cooling the fatty acid diester of polyethylene glycol obtained in step (a) to a temperature of 90-100° C., adding an N-acyl methyl taurate of Formula II, in powder form and stirring the reaction mixture to obtain a homogeneous mass,

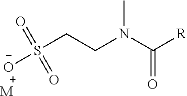

(where R═C₇ to C₁₇ and M⁺=Na⁺ or K⁺); and
c. cooling the homogeneous mass to a temperature of 70° C. to 80° C. and pouring the mass onto a double drum flaker to obtain the cold-processable viscosity-building composition in a flake form,
wherein the flakes obtained have thickness of maximum 1.0 mm, length of maximum 10 mm.

The ratio of fatty acid diester of PEG and N-cocoyl methyl taurate is maintained at the ratio of 0.7:0.3 to 0.9:0.1.

Preferably, the polyethylene glycol is selected from polyethylene glycol 150, polyethylene glycol 100 or mixture thereof.

Preferably, the fatty acid is selected from a long chain, saturated or unsaturated, fatty with C₁₀-C₁₈ linear or branched carbon chain.

Preferably the long chain fatty acid is selected from lauric acid, myristic acid, palmitic acid, stearic acid, coco fatty acids (a mixture of C₈ to C₁₈ fatty acids) or mixture thereof.

Preferably, the fatty acid diesters of polyethylene glycol are selected from polyethylene glycol 150 dilaurate, polyethylene glycol 150 dimyristate, polyethylene glycol 150 dipalmitate, polyethylene glycol 150 distearate, polyethylene glycol 100 distearate, polyethylene glycol 100 dilaurate or mixture thereof.

Preferably, the N-acyl methyl taurate is selected from sodium N-lauroyl methyl taurate, sodium N-cocoyl methyl taurate, or mixture thereof.

Preferably, the weight ratio of the fatty acid diesters of polyethylene glycol and the N-acyl methyl taurate is between 0.7:0.3 and 0.9:0.1. The weight ratio also allows the incorporation of high active viscosity building component, that is PEG 150 diester and other such derivatives of PEG, into the personal care cleansing formulations.

Most preferably the weight ratio of the fatty acid diesters of polyethylene glycol to the N-acyl methyl taurate is 0.8 to 0.2.

Step (a) of the one-pot process is an esterification step to prepare the fatty acid diester of polyethylene glycol. The step (a) is carried out at a temperature of 145-160° C. using a hypophosphorous acid as a catalyst. Other catalysts for esterification reaction known in art can also be used instead of hypophosphorous acid.

Preferably step (a) of preparing a diester of fatty acids and polyethylene glycol is carried out in a rection vessel with heating arrangement and equipped with overhead stirrer that are known in art.

Preferably, the step (b) includes cooling of the fatty acid diester of polyethylene glycol obtained in step (a) to 90 to 100° C. and adding the N-acyl methyl taurate.

Preferably the addition of the N-acyl methyl taurate is carried out at a temperature of 90-100° C. After the addition of the N-acyl methyl taurate, the reaction mixture is allowed to mix with sufficient stirring to obtain a homogeneous mass.

Preferably, the stirring of the reaction mixture is continued for 2 to 4 hours.

In an embodiment the step (c) of the one-pot process requires cooling of the homogeneous mass to a temperature of 70° C. to 80° C. and pouring the mass onto a double drum flaker to obtain the cold-processable viscosity-building composition of the present invention in a flake form.

In an embodiment, the double drum flakers comprise two drums that are allowed to spin in a direction opposite to each other. The drums are continuously rotating at a speed of 1-3 rotations per minute (RPM). In another embodiment, the distance between the two drums is maintained between 0.4 to 0.6 mm. In yet another embodiment the cooling water is allowed to pass inside the drum so that the surface of the rotating drums remains cool to aid the flaking of cold-processable viscosity building composition of the present invention.

Example 1a describes one-pot synthesis of PEG 150 distearate at 140-150° C., followed by mixing sodium N-lauroyl methyl taurate at 90-100° C. and flaking using double drum flaker to afford the off-white flakes with thickness of less than 1 mm. The whole process of Example 1a is completed in 8 h. The dispersion (1% in water) takes 15 minutes at 25° C. The same ‘one-pot’ process is repeated with PEG 150 dilaurate in Example 1b whereas Example 1c is done with PEG 150 distearate and sodium N-cocoyl methyl taurate. ‘One-pot’ process of Example 1d showcases pairing of PEG 100 distearate and sodium N-lauroyl methyl taurate.

The incorporation of fatty acid diester of polyethylene glycol to personal care formulations needs the heating step to prepare the personal care formulations. As described in the background, personal care formulations are manufactured on the scale of 1000 tons, which requires a tremendous amount of heat energy and sufficient time to prepare stable personal care formulations for mild cleansing. However, when the heating process is carried out, the personal care formulation must be cooled to room temperature, which would again require energy and time. This results in a significant increase in batch cycle time (or production time) as well as an increase in cost.

The cold-processable viscosity-building composition of the present invention eliminates the heating step required in the manufacture of personal care formulations/products and ultimately, it reduces the amount of energy required to manufacture the personal care formulations as well as recovers the batch cycle that is lost due to cooling of the heated personal care formulations. Hence, the viscosity-building composition of present invention provides the solution for two problems: the elimination of heating step and the reduction of batch cycle time and in the process economize it as well.

Personal Cleansing Formulations with Cold-Processable Thickening Composition of the Present Invention:

A personal cleansing formula with mild surfactants, namely, sodium cocoyl glutamate, sodium cocoyl isethionate and cocamido propyl betaine) is used to demonstrate comparative examples in Table 1 of Example 2. Entries IV, V and VI in Table 1 deploy viscosity-building compositions of the present invention ranging from 1.5 to 3.0% resulting into smooth transparent formulation at ambient temperature of 25° C., yielding viscosities ranging from 3500 cps to 7500 cps at pH closer to skin pH of 5.5. The same formulation of entry no II and III wherein PEG100 distearate and PEG150 distearate are attempted to disperse in the cleansing formula at room temperature but they are found to be ‘non-processable’. The experiment depicted in the entry number I of Table 1 is addition of PEG 150 distearate and sodium N-lauroyl methyl taurate separately to the cleansing formulation. However, at ambient temperature this comparative experiment is also observed to be ‘non-processable’. Therefore, to obtain the stable personal cleansing formulation of comparative formulations I, II and III, heating step is necessary. Examples 3, 4 and 5 depict use of viscosity-building compositions of the present invention of Example 1a in face wash (viscosity 4200 cps), shampoo (viscosity 9800 cps) and shower gel (viscosity 10,100 cps) formulations, all are based on mild, sulfate-free surfactants. Example 6 is a shampoo (viscosity 7600 cps) with viscosity building composition of Example 1c. Example 7 describes a ‘leave-on’ preparation like, a cream base which is an o/w emulsion. The viscosity building effect of the viscosity building composition of Example 1a is easily understood in this cold temperature (ambient temperature) processable cream formulation.

Cream formulation 1 of Table 2 of Example 7 forms a very low viscous cream (400 cps) which is with a small contribution of a thickener, the xanthan gum (0.5%). Addition of 2% viscosity building composition of Example 1a to the cream formulation 1 formula results in dramatic increase in the viscosity of the formulation to 4500 cps (Cream formulation 2 of Table 2).

In Cream formulation 3 of Table 2, thickener xanthan gum is removed and composition of Example 1a is added at 4% to boost the viscosity of the cream formulation to 27,000 cps.

Thus, it can be seen that the viscosity building composition of the present invention is performing not only for water-based cleansers but also in leave-on cream type o/w emulsions at ambient temperature.

Mild personal care cleansing products are gaining popularity in all the parts of the world. There have been continuous efforts by formulators to provide mild personal care products with desired properties. One important consumer attribute is viscosity profile.

A personal care product with high viscosity is highly accepted by consumers.

However, building viscosity in personal care products having mild surfactants has been a challenge. The present invention provides the solution to this challenge.

### Characteristics of the Composition of the Present Invention:

The cold-processable viscosity building composition of the present invention is in the flake form. It has a distinct flake morphology, characterized by thin, small, sheet-like shapes. The flakes vary slightly in size, ranging from 5-10 mm in length and may have 0.3-1.0 mm of thickness. Particularly the thickness of the flake of the viscosity building composition of the present invention is maximum of 1.0 mm. The flakes of PEG diester and taurates ought to be less than 1 mm, since going above this size result in an increase in the dispersion time and ultimately increase the batch cycle time. The weight of the flakes varies between 0.01-0.05 μm. These variations in dimensions result in different shapes, which help in achieving rapid dispersion in the aqueous solutions, ensuring complete dispersion in 15 to 30 minutes.

The size of the flakes of viscosity-building composition of the present invention are measured by standard measuring instruments known in the art.

The thickness of the flakes of viscosity building composition of the present invention are measured using a digital micrometer. The Anvil and Spindle of digital micrometer were set apart from each other with a distance of 1 mm. The flake particles were allowed to pass between the Anvil and Spindle. 25 flake particles easily pass between the Anvil and Spindle showing that the thickness of the flake is up to 1 mm maximum. In an embodiment the thickness of the flake according to the present invention is between 0.3 mm to 1.0 mm.

The length of the flakes of viscosity-building composition of the present invention is measured by measuring the two edges. Preferably, the length of about 15 to 30 particles is measured and an average length is calculated. Preferably, the length of the flake of viscosity-building composition of present invention is maximum 10 mm. Preferably the length of the flakes of viscosity building composition of the present invention between 4 to 10 mm.

### Application of Cold-Processable Viscosity Building Composition to Enhance Viscosity for Personal Care Formulations:

The cold-processable rheology modifying or viscosity building composition of the present invention can be incorporated into the personal cleansing formulations without the requirement of heating process. Also, the one-pot process of the present invention provides the cold-processable viscosity building composition with high active content of the viscosity modifying agents like those of PEG derivatives. This results in significant saving of both time and energy and in the process be economical.

The viscosity-building composition of the present invention is suitable for incorporating into all forms of personal care formulations meant for rinse-off or leave-on products (Example 7). Preferably personal care formulations are hand wash, body wash, face wash, shower gel, shampoo, and others form where viscosity is a critical parameter.

### Advantages of the Viscosity-Building Composition of the Present Invention:

Water-insoluble solid ingredients in personal care are usually made cold-processable by making them a part of liquid dispersion or emulsion (easy to pour at ambient temperature) that can be incorporated at an ambient temperature in personal cleansing formulations. This process has two serious drawbacks. Thus, making a liquified cold-processable ingredient (dispersion or emulsion) is a separate process that requires both energy and time. Secondly, the concentration of the solid ingredient that gets emulsified or dispersed is significantly low in concentration in pourable liquified composition.
Against this background, the process of making cold-processable viscosity-building composition of the present invention is carried out in ‘one-pot’ and avoids the separate processing step to convert Polyethylene glycol diester into cold-processable form. The process involves esterification between polyethylene glycol and fatty acid is carried out at 145° C. and then it is cooled to 90° C. At this temperature the anionic surfactant, N-acyl methyl taurate, in powder form is added while the polyethylene glycol diester mass is being cooled under stirring. The homogeneous mass is then flaked when the temperature reaches 70° C. to get the flakes of desired thickness. Thus, the ‘one-pot’ process to get the final cold-dispersible composition is very energy efficient compared to separate liquification processes reported in the literature (U.S. Pat. No. 6,165,955). This results in concentrated (80-90% active) and cold-processable viscosity-booster.
The main advantage of cold-processable rheology modifier of the present invention is that the significant saving on energy and high productivity of personal cleansing compositions are commonly made on huge scale. Because of the huge scale of operation, manufacturing of personal cleansing formulations is energy intensive since whole mass of several tonnes needs to be heated at least to 75 to 80° C. in most cases to mix the solid ingredients (pearlizers, antimicrobials, viscosity boosters etc.) that are always minor components of formulations (1.0 to 3.0% of the total) with water-based surfactant system. This challenge is acute when viscosity-building is attempted in compositions based on amino acid surfactants. The composition of the present invention is cold-processable viscosity-builder and incorporation of the same in personal care product results in total avoidance of energy expended and time spent on heating and cooling of entire batch. Thus, the use of cold-processable viscosity-builder reduces the batch cycle time and increases productivity of the end product i.e. personal care product, significantly.
The cold-processible rheology modifiers of the present invention allow conducting manufacture of shampoos and body washes at ambient temperature. This allows flexibility incorporating other thermolabile ingredients (peptides, proteins, vitamins, fragrances etc.) at any stage of manufacturing avoiding heat induced degradation, loss due to volatility and unwanted chemical reactions between the ingredients. Viscosity-building composition of the present invention also work well in ‘leave-on’ cream like emulsion (oil-in-water) which is a major formulation type for personal care products.
Yet another advantage of the one-pot process of the present invention is that this process is applicable to other rheology modifying ingredients such as polyglyceryl diesters, polylactylates of fatty acids that are not easily dispersible in water.

The following Examples provide non limiting illustration of working of the present invention.

### EXAMPLES

Sodium N-lauroyl methyl taurate (SLT), sodium N-cocoyl methyl taurate (SCT), cocamidopropyl betaine, Galsoft GLI 21 (sodium cocoyl glutamate and sodium cocoyl isethionate at a ratio of 2:1), Galguard Tetra, (a preservative blend (70% active) of phenoxyethanol, benzoic acid, capryloyl glycine and undecylenoyl glycine, and GalHueShield HCS (methoxycinnamidopropyl behendimonium chloride, as 30% aqueous solution) are commercial products from Galaxy Surfactants Ltd, India.

Galseer DermaGreen is procured from Galaxy Surfactants and is an oil-miscible skin active composition with prebiotic cocoyl proline and other two other emulsifiers, namely, glyceryl mono laurate and polyglyceryl-3 oleate.

Commercial grade of stearic acid used in the Examples is made up of 55% of palmitic acid and 45% of stearic acid is procured from Inter-Continental Oils and Fats PTE, Singapore.

Lauric acid is procured from Inter-Continental Oils and Fats PTE, Singapore. Polyethylene glycol (PEG)-150 and PEG-100, CAS No. 25322-68-3, are procured from Research Lab Fine Chem Industries (RLFCI), Mumbai, India.

The number ‘150’ in PEG 150 refers to repeating units of Ethylene Oxide. Thus PEG 150 corresponds PEG 6000 which has avg. mole wt. of 6000. Similarly, PEG 100 corresponds to PEG 4000 having avg. mol. Wt. of 4000.

Viscosities are measured using a Brookfield DV1 viscometer (Molel No. D 220) at room temperature, about 25° C., using spindle No. 4.

The thickness of the flakes according to the present invention is measured using a digital micrometer (Make: LABART, model No. L-5202N). The distance between the Anvil and Spindle is set to 1 mm and flakes are passed between the Anvil and Spindle.

The term “cold-processable” or “cold-dispersible” as used in the specifications, refers to the ease of processability or dispersiability of viscosity-building composition of the present invention in preparing personal cleansing formulations at ambient temperature.

The term “rheology modifying” and “viscosity building” are used interchangeably in the specifications and have same meaning, that is increasing the viscosity or providing the desired viscosity to the personal cleansing formulation.

### Example 1a: One-Pot Process of Preparing Viscosity-Building Composition of the Present Invention with PEG-150 Distearate and Sodium N-Lauroyl Methyl Taurate

### Step a.:

A mixture of PEG 150 (330 g, 0.05 gmol), stearic acid (27 g, 0.1 gmol) and hypophosphorous acid (0.35%) is heated in a reaction vessel at 140-150° C. with stirring under a steady nitrogen flow (alternatively under vacuum of 400 to 500 mm of Hg). The progress of the reaction is monitored by measuring the drop in acid value (7.5).

### Step b.:

The reaction mass of step (a) is cooled to a temperature of 90-100° C. To the reaction mixture sodium N-lauroyl methyl taurate (84.5 g) in powder, having particle size <200μ and 90% active, is added and heating of the mixture is continued at 95-100° C. for 2 hours with stirring to obtain a homogeneous mass.

### Step c:

The resulting homogeneous mass of step (b) is then cooled to 80° C. and poured onto a double drum flaker, wherein the rotation frequency of double drum is 2 rotations per minute and the distance between two drum is adjusted to 0.5 mm. The drums are supplied with cooling water which reduces the temperature of the drum surface and aids in flaking. The flakes obtain are off-white in color (428 g, 98% yield). The analysis of the viscosity building composition is presented in table below:

| Parameters | Observation | |
|---|---|---|
| Activity (based on mol. wt. 343) | 17.9 | |
| pH of 1% aq. Dispersion | 6.1 | |
| Time - 1% dispersion in water at 25° C. | 15 | minutes |
| Thickness | <1 | mm |
| Average length (for 20 measurements) | 7.4 | mm |

### Example 1b: One-Pot Process of Preparing of Viscosity-Building Composition of the Present Invention with PEG 150 Dilaurate and Sodium N-Lauroyl Methyl Taurate

### Step a:

A mixture of PEG 150 (330 g, 0.05 mole), lauric acid (20 g, 0.1 mole) and hypophosphorous acid (0.35%) is heated at 140-150° C. with good stirring under a steady nitrogen flow. The progress of the reaction is monitored by measuring the drop in acid value (7.2).

### Step b:

The reaction mixture of step (a) is cooled to a temperature of 90-100° C. To the reaction mixture of step (a) sodium N-lauroyl methyl taurate (86 g) in powder, particles size of less than 200μ and about 90% active, is added, and heating of the mixture is continued at 95-100° C. for 2 hours with stirring to obtain a homogeneous mass.

### Step c:

The resulting homogeneous mass of step (b) is then cooled to 80° C. and poured onto a double drum flaker, wherein the rotation frequency of double drum is 2 rotations per minute and the distance between two drum is adjusted to 0.5 mm. The drums are supplied with cooling water which reduces the temperature of the drum surface and aids in flaking. The flakes obtain are off-white in color. (420 g, yield 96.5%). The analysis of the viscosity building composition is presented in table below:

| Parameters | Observation | |
|---|---|---|
| Activity (based on mol. wt. 343) | 17.5 | |
| pH of 1% aq. dispersion | 6.0 | |
| Time - 1% dispersion in water at 25° C. | 17 | minutes |
| Thickness | <1 | mm |
| Average length (for 20 measurements) | 8.2 | mm |

### Example 1c: One-Pot Process for Preparation of Viscosity-Building Composition of the Present Invention with PEG 150 Distearate and Sodium N-Cocoyl Methyl Taurate

### Step a:

A mixture of PEG 150 (330 g, 0.05 mole), stearic acid (27 g, 0.1 mole) and hypophosphorous acid (0.35%) is heated at 140-150° C. with good stirring under a steady nitrogen flow. The progress of the reaction is monitored by drop in the acid value of the reaction (7.9).

### Step b:

The reaction mixture of step (a) is cooled to a temperature of 90-100° C. To the reaction mixture sodium N-cocoyl mehtyl taurate (84 g) in powder form, having particle size of less than 200μ and 90% active, is added, and heating of the mixture is continued at 95-100° C. for 2 hours with stirring to obtain a homogeneous mass.

### Step c:

The resulting homogeneous mass of step (b) is then cooled to 80° C. and poured onto a double drum flaker, wherein the rotation frequency of double drum is 2 rotations per minute and the distance between two drum is adjusted to 0.5 mm. The drums are supplied with cooling water which reduces the temperature of the drum surface and aids in flaking. The flakes obtain are off-white in color. (425 g, yield 98%). The analysis of the viscosity building composition is presented in table below:

| Parameters | Observation | |
|---|---|---|
| Activity (based on mol. wt. 343) | 17.7 | |
| pH of 1% aq. dispersion | 5.9 | |
| Time - 1% dispersion in water at 25° C. | 20 | minutes |
| Thickness | <1 | mm |
| Average length (for 20 measurements) | 8.4 | mm |

### Example 1d: One-Pot Process for Preparation of Viscosity-Building Composition of the Present Invention with PEG 100 Distearate and Sodium N-Lauroyl Methyl Taurate

A mixture of PEG 100 (240 g, 0.06 mole), stearic acid (32.16 g, 0.12 mole) and hypophosphorous acid (0.35%) is heated at 140-150° C. with good stirring under a steady nitrogen flow. The progress of the reaction is monitored via drop in the acid value (6.8).

### Step b:

The reaction mass of step (a) is cooled to a temperature of 90-100° C. To the reaction mixture sodium N-lauroyl methyl taurate (70 g) in powder, having particle size of less than 200μ and 90% active, is added and heating of the mixture is continued at 95-100° C. for 2 hours while stirring, to ensure uniform consistency and to obtain a homogeneous mass.

### Step c:

The resulting homogeneous mass of step (b) is then cooled to 80° C. and poured onto a double drum flaker, wherein the rotation frequency of double drum is 2 rotations per minute and the distance between two drum is adjusted to 0.5 mm. The drums are supplied with cooling water which reduces the temperature of the drum surface and aids in flaking. The flakes obtain are off-white in color. (338 g, 98% yield).

The analysis of the viscosity building composition is presented in table below:

| Parameters | Observation | |
|---|---|---|
| Activity (based on mol. wt. 343) | 17.4 | |
| pH of 1% aq. Dispersion | 6.0 | |
| Time - 1% dispersion in water at 25° C. | 20 | minutes |
| Thickness | <1 | mm |
| Average length (for 20 measurements) | 7.2 | mm |

### Example 2: Personal Cleansing Formulations with the Viscosity-Building Composition of the Present Invention and Comparative Formulations

TABLE 1
Personal cleansing formulation with mild surfactants along with different viscosity-building
compositions according to the present invention as well as comparative formulations:

| | | | | (IV) | (V) | (VI) |
|---|---|---|---|---|---|---|
| | | | | w/w % | w/w % | w/w % |
| INCI / | (I) | (II) | (III) | Formulations with the | | |
| Chemical | w/w % | w/w % | w/w % | viscosity-building composition | | |
| name | Comparative formulations | | | of the present invention | | |
| Distilled Water | q.s. to | q.s. to | q.s. to | q.s. to | q.s. to | q.s. to |
| | 100% | 100% | 100% | 100% | 100% | 100% |
| Sodium | 15.0% | 15.0% | 15.0% | 15.0% | 15.0% | 15.0% |
| Cocoyl | | | | | | |
| glutamate | | | | | | |
| (Galsoft SCGL | | | | | | |
| (30%) | | | | | | |
| Sodium cocoyl | 5.0% | 5.0% | 5.0% | 5.0% | 5.0% | 5.0% |
| glutamate and | | | | | | |
| sodium cocoyl | | | | | | |
| isethionate | | | | | | |
| (2:1, flake) | | | | | | |
| (Galsoft GLI | | | | | | |
| 21 (F)) | | | | | | |
| Galaxy CAPB | 13.0% | 13.0% | 13.0% | 13.0% | 13.0% | 13.0% |
| (35% solids) | | | | | | |
| Glycerin | 2.0% | 2.0% | 2.0% | 2.0% | 2.0% | 2.0% |
| Sodium N- | 0.2% | — | — | — | — | — |
| lauroyl methyl | | | | | | |
| taurate | | | | | | |
| PEG 100 | — | 2.4% | — | — | — | — |
| Distearate | | | | | | |
| PEG 150 | 1% | — | — | — | — | — |
| Distearate | | | | | | |
| PEG 150 | — | — | 1.5% | — | — | |
| Dilaurate | | | | | | |
| PEG 150 | — | — | — | 2% | — | — |
| Dilurate:SLT | | | | | | |
| (80:20) - | | | | | | |
| Example 1b | | | | | | |
| PEG 150 | — | — | — | — | — | 1.5% |
| Distearate:SLT | | | | | | |
| (80:20) - | | | | | | |
| Example 1a | | | | | | |
| PEG 100 | — | — | — | — | 3% | — |
| Distearate:SLT | | | | | | |
| (80:20) - | | | | | | |
| Example 1d | | | | | | |
| GLDA | 0.2% | 0.2% | 0.2% | 0.2% | 0.2% | 0.2% |
| (Tetrasodium | | | | | | |
| glutamate | | | | | | |
| diacetate) | | | | | | |
| Appearance | Hazy low | Hazy low | Hazy low | Clear | Clear | Clear |
| | viscous | viscous | viscous | viscous | viscous | viscous |
| | liquid | liquid | liquid | liquid | liquid | liquid |
| pH - adjusted | NA | NA | NA | 5.42 | 5.40 | 5.38 |
| with Citric acid | | | | | | |
| (50% aq. sol) | | | | | | |
| Processability | Not | Not | Not | Processable | Processable | Processable |
| of the viscosity | processable | processable | processable | | | |
| building | | | | | | |
| compositions | | | | | | |
| at room | | | | | | |
| temperature | | | | | | |
| Viscosity (cPc) | NA | NA | NA | 3500 | 3400 | 7200 |

The formulations IV, V and VI with the viscosity-building composition of the present invention, are prepared without the requirement of the heating step. Briefly, the viscosity modifying composition of the present invention, as per example 1, is dispersed into 20 to 40% of total required water quantity. The viscosity modifying composition is dispersed within 15 to 30 minutes and all the ingredients are added except fragrance and color. The pH is adjusted between 5 to 6 and color and fragrances were added.

However, the comparative formulations I, II and III, without the viscosity-building composition of the present invention but having Sodium N-lauroyl methyl taurate and PEG fatty acid ester added separately, were found to be not processable at room temperature and do not provide the desired personal care formulation. However, when these formulations are heated to around 70° C. the formulations appear to be processable and give a clear formulation. Hence, this indicates the requirement of heating step for preparation of personal care formulations of comparative examples I, II and III.

### Example 3: Facewash Formulation with the Viscosity-Building Composition of Example 1a

| INCI/Chemical name | (% w/w) |
|---|---|
| Water (Aqua) | To make |
| | 100.00 |
| Phenoxyethanol (and) Benzoic | 1.20 |
| Acid (and) Capryloyl Glycine | |
| (and) Undecylenoyl Glycine - | |
| (Galguard Tetra) | |
| PEG 150 Distearate:SLT | 01.25 |
| (80:20) - Example 1a | |
| Sodium Cocoyl Isethionate | 06.00 |
| (and) Disodium Cocoyl | |
| Glutamate (Galsoft GLI 21) | |
| Cocamidopropyl Betaine | 15.00 |
| Tetrasodium glutamate diacetate | 00.10 |
| Color & Fragrance | Qs |
| NaOH (40% w/w solution) | To adjust |
| | pH 5.7 |

Procedure: The formulation is prepared by first dissolving the viscosity-building composition as per Example 1a in 30 ml of water to achieve homogeneous dispersion. Following this, Galsoft GLI 21(F) is added, the remaining water and other specified ingredients are added, and the mixture is thoroughly mixed to achieve uniformity. The pH is then adjusted to between 5.7 using a 40% w/w NaOH solution followed by addition of color and fragrance.

Viscosity: 4200 cP (#04, rpm 10, Torque-58% at 23.4° C.)

### Example 4: Shampoo Formulation with the Viscosity-Building Composition of Example 1a

| INCI/Chemical name | (% w/w) |
|---|---|
| Water (Aqua) | To make |
| | 100.00 |
| Phenoxyethanol (and) Benzoic | 1.20 |
| Acid (and) Capryloyl Glycine | |
| (and) Undecylenoyl Glycine | |
| (Galguard Tetra) | |
| Methoxycinnamidopropyl | 01.00 |
| Behendimonium Chloride | |
| (GalHueShiled HCS) | |
| PEG 150 Distearate:SLT | 01.25 |
| (80:20) - Example 1a | |
| Sodium Cocoyl Isethionate | 07.00 |
| (and) Disodium Cocoyl | |
| Glutamate (Galsift GLI 21) | |
| Cocamidopropyl Betaine | 24.00 |
| Tetrasodium glutamate diacetate | 00.10 |
| Color & Fragrance | Qs |
| NaOH (48% w/w solution) | To adjust |
| | pH 5.9 |

Procedure: The formulation is prepared by first dissolving the viscosity-building composition as per Example 1a in 30 ml of water to achieve a homogeneous dispersion. Following this, Galsoft GLI 21(F) is added, the remaining water and other specified ingredients are added, and the mixture is thoroughly mixed to achieve uniformity. The pH is then adjusted to between 5.9 using a 48% w/w NaOH solution followed by addition of color and fragrance.

Viscosity: 9800 cP (#04, rpm 10, Torque-58% at 23.4° C.).

### Example 5: Shower Gel Formulation with the Viscosity-Building Composition of Example 1a

| INCI/Chemical name | (% w/w) |
|---|---|
| Water (Aqua) | To make |
| | 100.00 |
| Phenoxyethanol (and) Benzoic | 1.20 |
| Acid (and) Capryloyl Glycine | |
| (and) Undecylenoyl Glycine | |
| (Galguard Tetra) | |
| PEG 150 Distearate:SLT | 01.25 |
| (80:20) - Example 1a | |
| Sodium Cocoyl Isethionate | 07.00 |
| (and) Disodium Cocoyl | |
| Glutamate (Galsoft GLI 21) | |
| Cocamidopropyl Betaine | 22.00 |
| Tetrasodium glutamate diacetate | 00.10 |
| Color & Fragrance | Qs |
| NaOH (48% w/w solution) | To adjust |
| | pH 5.6 |

Procedure: The viscosity-building composition of Example 1a is dissolved in water. After homogeneous dispersion, all ingredients, except color and fragrance, are added and mixed well to get homogeneous mixture. pH is adjusted with the NaOH (48% solution) to 5.6 followed by addition of color and fragrance.

Viscosity: 10100 cP (#04, rpm 10, Torque-57% at 23.4° C.).

### Example 6: Shampoo Formulations with the Viscosity-Building Composition of Example 1c

| INCI/Chemical name | (% w/w) |
|---|---|
| Water (Aqua) | To make |
| | 100.00 |
| Phenoxyethanol (and) Benzoic | 1.20 |
| Acid (and) Capryloyl Glycine | |
| (and) Undecylenoyl Glycine | |
| (Galguard Tetra) | |
| Methoxycinnamidopropyl | 01.00 |
| Behendimonium Chloride | |
| (GalHueShiled HCS) | |
| PEG 150 Disterate:SCT | 3.0 |
| (80:20) - Example 1c | |
| Sodium Cocoyl Isethionate | 07.00 |
| (and) Disodium Cocoyl | |
| Glutamate (Galsoft GLI 21) | |
| Cocamidopropyl Betaine | 24.00 |
| Tetrasodium glutamate diacetate | 00.10 |
| Color & Fragrance | Qs |
| NaOH (48% w/w solution) | To adjust |
| | pH 5.5 |

Procedure: The formulation is prepared by first dissolving the viscosity-building composition as per Example 1c in 30 ml of water to achieve a homogeneous dispersion. Following this, Galsoft GLI 21(F) is added, the remaining water and other specified ingredients are added, and the mixture was thoroughly mixed to achieve uniformity. The pH is then adjusted to 5.5 using a 48% w/w NaOH solution followed by addition of color and fragrance.

Viscosity: 7600 cP (#04, rpm 10, Torque-58% at 23.4° C.).

### Example 7: Cold-Processable Cream Formulation with the Composition of Example 1a

TABLE 2
Cream formulation with the composition of
Example 1a and comparative formulations.

| INCI/Chemical | Cream | Cream | Cream |
|---|---|---|---|
| name | formulation 1 | formulation 2 | formulation 3 |
| Water | To make | To make | To make |
| | 100.00 | 100.00 | 100.00 |
| Xanthan gum | 00.50 | 00.50 | — |
| Phenoxyethanol (and) | 01.00 | 01.00 | 01.00 |
| Benzoic Acid (and) | | | |
| Capryloyl Glycine | | | |
| (and) Undecylenoyl | | | |
| Glycine - Galguard | | | |
| Tetra | | | |
| Glycerin | 04.00 | 04.00 | 04.00 |
| Propylene Glycol | 03.00 | 03.00 | 03.00 |
| Caprylic/Capric | 05.00 | 05.00 | 05.00 |
| Triglyceride - | | | |
| GalMOL CCT | | | |
| Cocoyl proline (and) | 04.00 | 04.00 | 04.00 |
| glyceryl mono laurate | | | |
| (and) polyglyceryl-3 | | | |
| oleate - Galseer | | | |
| DermaGreen | | | |
| Example 1a | — | 02.00 | 04.00 |
| pH | 5.7 | 5.7 | 5.7 |
| Viscosity (RV 04) | 400 cPs | 4500 cPs | 27000 cPs |

The cold-processability of the viscosity building composition of the present invention is also demonstrated in the cream formulations. Here, the incorporation of the viscosity building composition of the present invention as per example 1a, results into a cream formulation 2 and 3 that shows drastic increase in viscosity compared to formulation 1. The formulation 3 does not even have xanthan gum, the known viscosity modifier.

We claim:

1. A one-pot process of preparing a cold-processable viscosity-building composition comprising steps of:
a. heating polyethylene glycol with a fatty acid at a temperature of 140 to 150° C. to prepare a fatty acid diester of polyethylene glycol of Formula I, 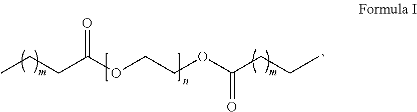
wherein m=9 to 15 and n=100 to 150;
b. cooling the fatty acid diester of polyethylene glycol obtained in step (a) to a temperature of 90 to 100° C., adding an N-acyl methyl taurate, of Formula II, in powder form and stirring the reaction mixture to obtain a homogeneous mass, 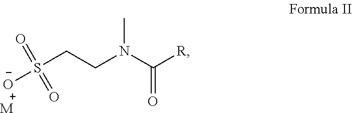
wherein R═C₇ to C₁₇ and M⁺=Na⁺ or K⁺;
c. cooling the homogeneous mass to a temperature of 70° C. to 80° C. and pouring onto a double drum flaker to obtain the cold-processable viscosity-building composition in a flake form,
wherein thickness of the flake is maximum 1.0 mm and length of the flake is maximum 10 mm, and
wherein 1% homogeneous dispersion of the flake in water is formed in 15 to 30 minutes of stirring at 25° C.

2. The one-pot process as claimed in claim 1 wherein the weight ratio of the fatty acid diester of polyethylene glycol to the N-acyl methyl taurate is between 0.7:0.3 and 0.9:0.1.

3. The one-pot process as claimed in claim 1 wherein, the fatty acid is selected from lauric acid, myristic acid, palmitic acid, stearic acid or mixture thereof.

4. The one-pot process as claimed in claim 1 wherein, the N-acyl methyl taurate is selected from sodium N-lauroyl methyl taurate, sodium N-cocoyl methyl taurate or mixture thereof.

5. The one-pot process as claimed in claim 1 wherein, the fatty acid diester of polyethylene glycol is selected from polyethylene glycol 150 dilaurate, polyethylene glycol 150 dimyristate, polyethylene glycol 150 dipalmitate, polyethylene glycol 150 distearate, polyethyleneglycol 100 distearate or mixture thereof.

6. A cold-processable viscosity-building composition comprising:
a. a fatty acid diester of polyethylene glycol of Formula I 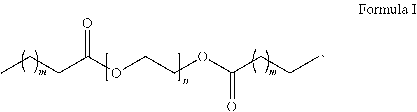
wherein m=9 to 15 and n=100 to 150; and
b. an N-acyl methyl taurate Formula II 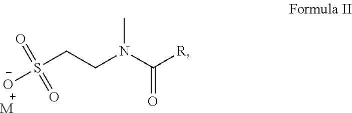
wherein R═C₇ to C₁₇ and M⁺=Na⁺ or K⁺,
wherein the weight ratio of the fatty acid diester of polyethylene glycol to the N-acyl methyl taurate is between 0.7:0.3 and 0.9:0.1,
wherein the viscosity-building composition is in a flake form,
wherein the thickness of the flake is maximum 1.0 mm and length of the flake is maximum 10 mm, and
wherein 1% homogeneous dispersion of the cold-processable viscosity-building composition in water is formed in 15 to 30 minutes of stirring at 25° C.

7. The cold-processable viscosity-building composition as claimed in claim 5 wherein, the N-acyl methyl taurate is selected from sodium N-lauroyl methyl taurate, sodium N-cocoyl methyl taurate or mixture thereof.

8. The cold-processable viscosity-building composition as claimed in claim 5 wherein, the fatty acid diester of polyethylene glycol is selected from polyethylene glycol 150 dilaurate, polyethylene glycol 150 dimyristate, polyethylene glycol 150 dipalmitate, polyethylene glycol 150 distearate, polyethylene glycol 100 distearate or mixture thereof.

9. A personal care cleansing formulation comprising the viscosity building composition as claimed in claim 5.
